(19) United States
(12) Patent Application Publication
(10) Pub. No.: US 2022/0202303 A1
(43) Pub. Date: Jun. 30, 2022

(54) Heart Rate Module, and Electronic Device for Collecting Heart Rate

(71) Applicant: Weifang Goertek Microelectronics Co. Ltd., Weifang, Shandong (CN)

(72) Inventors: Wentao Wang, Weifang, Shandong (CN); Huabin Fang, Weifang, Shandong (CN); Dexin Wang, Weifang, Shandong (CN)

(73) Assignee: Weifang Goertek Microelectronics Co. Ltd., Weifang, Shandong (CN)

(21) Appl. No.: 17/606,524

(22) PCT Filed: Dec. 6, 2019
(86) PCT No.: PCT/CN2019/123553

§ 371 Date: Oct. 26, 2021

(30) Foreign Application Priority Data
Apr. 26, 2019 (CN) 2019 10342911.5

(51) Int. Cl. A61B 5/024; A61B 5/00; A61B 5/026; A61B 5/1455

(52) U.S. Cl. CPC A61B 5/02433; A61B 5/72; A61B 2562/0238; A61B 5/14552; A61B 2560/0214; A61B 5/0261

(57) ABSTRACT

Disclosed are a heart rate module and an electronic device for collecting heart rate, the heart rate module includes a substrate, and a first light wave emitting unit, a second light wave emitting unit, a first optical sensor chip, and a second optical sensor chip provided on the substrate; the substrate is also provided thereon with an isolation grating wall separating the first light wave emitting unit, the second light wave emitting unit, the first optical sensor chip, and the second optical sensor chip from each other; the isolation grating wall together with the substrate enclose respectively a first accommodating cavity for accommodating the first light wave emitting unit, a second accommodating cavity for accommodating the second light wave emitting unit, a third accommodating cavity for accommodating the first optical sensor chip, and a fourth accommodating cavity for accommodating the second optical sensor chip.

### CROSS-REFERENCE TO RELATED APPLICATION

This application is a National Stage of International Application No. PCT/CN2019/123553, filed on Dec. 6, 2019, which claims priority to Chinese Patent Application No. 201910342911.5, filed on Apr. 26, 2019, both of which are hereby incorporated by reference in its entirety.

### TECHNICAL FIELD

The present disclosure relates to the field of optical sensor, and more specifically, to a heart rate module and an electronic device for collecting heart rate.

### BACKGROUND

Nowadays, due to various factors such as work pressure and unreasonable diet pattern, cardiovascular and cerebrovascular diseases such as hypertension and coronary heart disease have gradually become common and frequently-occurring diseases in clinical medicine. Moreover, cardiovascular and cerebrovascular diseases no longer only occur in the middle-aged and elderly people, but gradually tend to be younger and have aroused widespread concern from various circles of society. In fact, most of the cardiovascular and cerebrovascular diseases are chronic sudden diseases, and can only be controlled but cannot be cured, which requires a patient to go to a hospital for regular checkups and take related drugs for a long time. However, even so, occurrence of emergencies cannot be completely avoided. In order to avoid emergencies as much as possible, it is necessary to monitor the patient's heart rate changes, blood pressure and other parameters in real time so as to find a problem in time, so that corresponding treatment measures can be quickly taken to avoid sudden occurrence of dangerous situations.

In recent years, with increasing living standards, many people begin to attach importance to exercise to maintain their health and prevent diseases through exercise. However, during exercise, it is necessary to control the amount of exercise reasonably, that is, not to exercise too much; otherwise it will cause physical injury. In response to this situation, heart rate detection devices have been introduced on the market, and can determine a suitable exercise plan by detecting the heart rate. Wherein, smart bracelets and smart watches are currently more common portable heart rate detection devices, are generally recognized by consumers because of their compact size, convenient carrying, and normal use during exercise, and can detect the user's heart rate changes in real time after being worn, which is very convenient to use.

However, in terms of the prior art, regardless of a separation scheme or an integrated module scheme, most of the heart rate detection device have the problem of margin loss in the light intensity of the LED. Specifically: in the existing heart rate sensor, the light emitted by the LED is scattered around, and the light scattered to obstacles will be absorbed and consumed by the obstacles, which will reduce the light incident on human skin or blood for detection and thus affect the accuracy of the final detection. It can be seen that it is very necessary to reasonably adjust the internal structure and light path of the existing heart rate modules to optimize the light intensity of the LED.

### SUMMARY

An object of the present disclosure is to provide a new technical solution of a heart rate module.

According to a first aspect of the present disclosure, a heart rate module is provided, including a substrate as well as a first light wave emitting unit, a second light wave emitting unit, a first optical sensor chip, and a second optical sensor chip provided on the substrate; the substrate is also provided thereon with an isolation grating wall separating the first light wave emitting unit, the second light wave emitting unit, the first optical sensor chip, and the second optical sensor chip from each other;

the isolation grating wall together with the substrate enclose respectively a first accommodating cavity for accommodating the first light wave emitting unit, a second accommodating cavity for accommodating the second light wave emitting unit, a third accommodating cavity for accommodating the first optical sensor chip, and a fourth accommodating cavity for accommodating the second optical sensor chip; the first accommodating cavity and the second accommodating cavity both have a pattern draft on peripheral inner walls thereof, and provided on all or part of surfaces of the inner walls thereof with non-conductive reflective coatings.

Optionally, the reflective coatings are made of Ti₃0₅ material or SiO₂ material or non-conductive high-aluminum reflective film.

Optionally, the reflective coatings have a thickness no greater than 1 μm.

Optionally, the pattern draft is ≥45°.

Optionally, the first optical sensor chip and the first light wave emitting unit have a distance of 6 to 10 mm therebetween;

the second optical sensor chip and the second light wave emitting unit have a distance of 2.3 to 3.2 mm therebetween.

Optionally, the first optical sensor chip and the first light wave emitting unit have a distance of 9 mm therebetween; the second light wave emitting unit and the second optical sensor chip are provided between the first optical sensor chip and the first light wave emitting unit, the second light wave emitting unit is close to the first optical sensor chip, the second optical sensor chip is close to the first light wave emitting unit, and the second optical sensor chip and the second light wave emitting unit have a distance of 2.5 mm therebetween.

Optionally, the first light wave emitting unit includes two red LED chips and one infrared LED chip, and the two red LED chips and the one infrared LED chip are distributed in a straight line, and the infrared LED chip is located between the two red LED chips.

Optionally, the second light wave emitting unit includes three green LED chips which are distributed in a straight line.

Optionally, the isolation grating wall is also provided thereon with light-transmitting windows that allow the first accommodating cavity, the second accommodating cavity, the third accommodating cavity, and the fourth accommodating cavity to be in communication with the outside.

Optionally, the heart rate module further comprises an analog front end and a power management unit provided on the substrate; the first optical sensor chip and the second optical sensor chip are respectively mounted on the power management unit and the analog front end.

According to a second aspect of the present disclosure, a heart rate module is provided, including any one of the above-mentioned heart rate modules.

The heart rate module provided by the embodiments of the present disclosure packages various components together reasonably, reduces the space occupied by the various components of the whole machine, and reduces the volume of the heart rate module, that is, realizing the miniaturization of the heart rate module. Moreover, by reasonably arranging the internal structure of the module and adjusting the optical path, the present disclosure can effectively avoid scattering of the light emitted by the LED. That is, the present disclosure can gather the light emitted by the LED in a large amount, so that more light can be emitted to the human skin or blood, thus contributing to improvement on the accuracy of detection results.

Other features and advantages of the present disclosure will become apparent from the following detailed description of exemplary embodiments of the present disclosure with reference to the accompanying drawings.

### BRIEF DESCRIPTION OF THE DRAWINGS

The accompanying drawings, which constitute a part of the description, illustrate embodiments of the present disclosure and, together with the description thereof, serve to explain the principles of the present disclosure.

FIG. 1 is a schematic diagram of the structure of a heart rate module of the present disclosure.

### DESCRIPTION OF REFERENCE SIGNS

1—red LED chip, 2—infrared LED chip, 3—analog front end, 4—power management unit, 5—green LED chip, 6—isolation grating wall, 7—first optical sensor chip, 8—second optical sensor chip, 9—reflective coatings

### DETAILED DESCRIPTION

Various exemplary embodiments of the present disclosure will now be described in detail with reference to the accompanying drawings. It should be noted that the relative arrangement, numerical expressions and numerical values of the components and steps set forth in these examples do not limit the scope of the disclosure unless otherwise specified.

The following description of at least one exemplary embodiment is in fact merely illustrative and is in no way intended as a limitation to the present disclosure and its application or use.

Techniques, methods, and apparatus known to those of ordinary skill in the relevant art may not be discussed in detail but where appropriate, the techniques, methods, and apparatus should be considered as part of the description.

Among all the examples shown and discussed herein, any specific value should be construed as merely illustrative and not as a limitation. Thus, other examples of exemplary embodiments may have different values.

It should be noted that similar reference numerals and letters denote similar items in the accompanying drawings, and therefore, once an item is defined in a drawing, and there is no need for further discussion in the subsequent accompanying drawings.

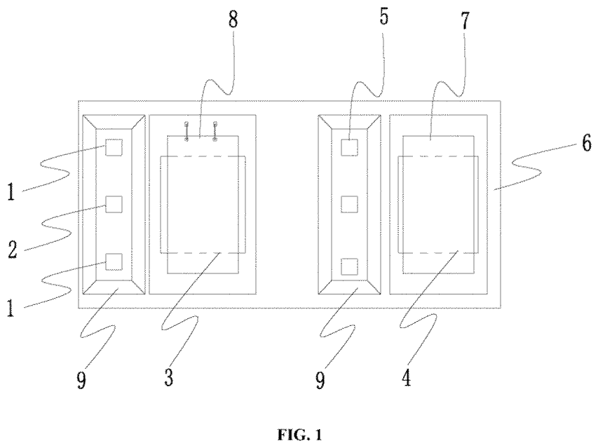

Referring to FIG. 1, a heart rate module is provided by an embodiment of the present disclosure, and includes a substrate (not shown in FIG. 1) as well as a first light wave emitting unit, a second light wave emitting unit, a first optical sensor chip 7, and a second optical sensor chip 8 arranged on the substrate. Wherein the substrate can be a circuit board well known to those skilled in the art, and can be provided with a circuit layout of the heart rate module therein in advance. The first optical sensor chip 7 and the second optical sensor chip 8 can be photodiodes PD well known to those skilled in the art, and can be used to convert optical signals into electrical signals, which will not be described in detail in the present disclosure.

Referring to FIG. 1, the first light wave emitting unit of the present disclosure includes two red LED chips 1 and one infrared LED chip 2, and the infrared LED chip 2 is distributed in a straight line with the two red LED chips 1 and is located between the two red LED chips 1. In other words, two red LED chips 1 and one infrared LED chip 2 are arranged in strips on the substrate. The first light wave emitting unit is configured to emit red light waves (the two red LED chips 1) and infrared light waves (the one infrared LED chip 2). The red light waves and infrared light waves emitted by the first light wave emitting unit of the present disclosure can be used to test blood oxygen saturation data, heart rate data, and the like. Wherein the two red LED chips 1 and one infrared LED chip 2 can be mounted on the substrate in a manner well known in the art, for example, can be fixedly provided on the substrate by mounting. Moreover, unlike a traditional light wave emitting unit that uses a single red LED chip, two red LED chips are used in the present disclosure, which can not only make intensity of the emitted red light high, but also effectively increase the detection range. In fact, when the single red LED chip is used, the detection range is relatively small, and especially during exercise, the heart rate module is prone to deviation; at this point there may be no red light waves feedback or a small amount of red light waves feedback, which will eventually affect the accuracy of the detection result.

Referring to FIG. 1, the second light wave emitting unit of the present disclosure includes three green LED chips 5 which are distributed together in a straight line. The second light wave emitting unit is configured to emit green light waves. The green light waves emitted by the second light wave emitting unit of the present disclosure can also be used to test the heart rate. Specifically, the green light waves emitted by the green LED chips 5 can cooperate with the second light wave emitting unit to measure changes in the density of blood flowing in blood vessels, and the heart rate data can be tested after further calculations. Wherein, the three green LED chips 5 can be mounted on the substrate in a manner well known in the art, for example, can be fixedly provided on the substrate by mounting. Unlike a traditional light wave emitting unit that uses a single green LED chip, three green LED chips 5 are used in the present disclosure to increase the intensity of the emitted green light, so that more green light signals are reflected from the blood. In the present disclosure, three green LED chips are arranged in strips, which can effectively increase the detection range. Even when the heart rate module is shifted due to exercise or other reasons, more green signals can be feedback, which can effectively prevent a single green LED chip from causing undetected signals.

The first optical sensor chip 7 of the present disclosure is configured to receive red light waves and infrared light waves reflected from the skin or blood. The second optical sensor chip 8 of the present disclosure is configured to receive green light waves reflected from the skin or blood. Moreover, the first optical sensor chip 7 and the second optical sensor chip 8 are also configured to simultaneously receive different light waves. Specifically: when the light emitted by the first light wave emitting unit and the second light wave emitting unit is emitted into human skin or blood, the light reflected by the skin or blood is received by the first optical sensor chip 7 and the second optical sensor chip 8 respectively, and the first optical sensor chip 7 and the second optical sensor chip 8 can send different control signals according to the intensity of light. Wherein, both the first optical sensor chip 7 and the second optical sensor chip 8 can be mounted on the substrate in a manner well known in the art, for example, can be fixedly provided on the substrate by mounting. Compared with a traditional heart rate module or heart rate sensor, the heart rate module of the present disclosure uses two optical sensor chips which can increase the detection range of the reflected light waves, and can also receive the red light waves/infrared light waves and green light waves reflected from the skin or blood respectively, can test blood oxygen saturation data and heart rate data, etc. respectively, thus achieving real-time detection.

In the heart rate module of the present disclosure, the substrate is also provided thereon with an isolation grating wall 6 for isolating the first light wave emitting unit, the second light wave emitting unit, the first optical sensor chip 7, and the second optical sensor chip 8. Wherein, the isolation grating wall 6 is made of opaque materials, and is used to optically isolate the first light wave emitting unit, the second light wave emitting unit, the first optical sensor chip 7, and the second optical sensor chip 8, which can effectively prevent the light emitted by the first light wave emitting unit from being directly received by the first optical sensor chip 7 and prevent the light emitted by the second light wave emitting unit being received by the second optical sensor chip 8, such that the first light wave emitting unit and the first optical sensor chip 7, and the second light wave emitting unit and the second optical sensor chip 8 will not interfere with each other, thus contributing to the accuracy of the test.

In addition, the isolation grating wall 6 of the present disclosure together with the substrate enclose respectively a first accommodating cavity for accommodating the first light wave emitting unit, a second accommodating cavity for accommodating the second light wave emitting unit, a third accommodating cavity for accommodating the first optical sensor chip 7, and a fourth accommodating cavity for accommodating the second optical sensor chip 8. Moreover, the isolation grating wall 6 is also provided with light-transmitting windows thereon for communicating the first, second, third, and fourth accommodating cavities with the outside world. At this time, the light waves emitted by the first light wave emitting unit and the second light wave emitting unit can be emitted to the outside through the light-transmitting windows on the isolation grating wall 6 so as to be received by the human skin or blood, and the first optical sensor chip 7 and the second optical sensor chip 8 can respectively receive light waves that are reflected from the skin or blood and enter through the corresponding light-transmitting windows.

It should be noted that the isolation grating wall 6 and the substrate can be combined together by bonding. Of course, the isolation grating wall 6 and the substrate can also be combined into a whole by ultrasonic welding, which is not limited in the present disclosure.

Wherein, referring to FIG. 1, the first and second accommodating cavities have a pattern draft on peripheral inner walls thereof, and are provided on all or a part of the inner wall surfaces thereof with non-conductive reflective coatings 9. The area of the reflective coatings 9 can be flexibly adjusted according to actual conditions, and this is not limited in the present disclosure. The reflective coatings 9 can make the inner walls of the first and the second accommodating cavities form a bright reflective surface (that is, a light-gathering surface), and when the first light wave emitting unit and the second light wave emitting unit emit light waves, the reflective coatings 9 can produce a good light-gathering effect. In other words, by changing the design of light paths, a good light-gathering effect is obtained. Through the pattern draft and the coatings process, the present disclosure can increase the amount of light emitted by the first light wave emitting unit and the second light wave emitting unit, and reduce the loss of light, so that more light can be emitted to human skin or blood, thus contributing to improvement on the accuracy of heart rate detection results.

Wherein, the reflective coatings 9 can be made of non-conductive reflective materials. For example, the reflective coatings 9 can be made of Ti305 material or SiO2 material. Of course, the reflective coatings 9 can also be made of other materials well known to those skilled in the art, such as a non-conductive high-aluminum reflective film, and this is not limited in the present disclosure.

Wherein, the reflective coatings 9 should have a thickness no greater than 1 μm, that is, the thickness of the reflective coatings 9 should be less than or equal to 1 μm. The thickness of the reflective coatings 9 should not be too large, otherwise it will affect the firmness of the combination of the reflective coatings 9 with the inner walls of the first and the second accommodating cavities, and increase production costs.

Wherein, the first and second accommodating cavities both have a pattern draft on peripheral inner walls thereof, and the patter draft can be controlled to be ≥45° and can be flexibly adjusted according to actual situations, so as to obtain a good light-gathering effect. For example, the patter draft can be 45°, 50°, 60°, 65°, 70°, 75°, 80°, etc., and can be flexibly adjusted according to actual conditions, which is not limited in the present disclosure. In addition, if 45° cannot be reached due to the size, then the pattern draft does not need to be specially set, as long as there is an angle.

In addition, the substrate of the present disclosure may also fixedly combined with a first transparent colloid, a second transparent colloid, a third transparent colloid, and a fourth transparent colloid. Specifically: the first light wave emitting unit (the two red LED chips 1 and the one infrared LED chip 2) can be fixedly provided on the substrate by the first transparent colloid; the second light wave emitting unit (the three green LED chips 5) can be fixedly provided on the substrate by the second transparent colloid; the first optical sensor chip 7 can be fixedly provided on the substrate by the third transparent colloid; and the second optical sensor chip 8 can be fixedly provided on the substrate by the fourth transparent colloid. That is, the first transparent colloid, the second transparent colloid, the third transparent colloid, and the fourth transparent colloid can respectively protect the first light wave emitting unit, the second light wave emitting unit, the first optical sensor chip 7, and the second optical sensor chip 8 without affecting the functions of various components (for example, emitting light waves outward, or receiving light waves reflected back), so as to facilitate the completion of the test. It should be noted that the first transparent colloid, the second transparent colloid, the third transparent colloid, and the fourth transparent colloid are respectively injected into the first, second, third and fourth accommodating cavities through the corresponding light-transmitting windows, and after the above-mentioned colloid is finally cured, the first light wave emitting unit, the second light wave emitting unit, the first optical sensor chip 7, and the second optical sensor chip 8 can be fixedly provided on the substrate.

Of course, the first light wave emitting unit, the second light wave emitting unit, the first optical sensor chip 7, and the second optical sensor chip 8 can also be directly fixed on the substrate. At this point, the respective light-transmitting windows of the first, second, third and fourth accommodating cavities are respectively provided with transparent glass. In this way, the first light wave emitting unit, the second light wave emitting unit, the first optical sensor chip 7, and the second optical sensor chip 8 can also be protected. However, since the transparent glass has a certain thickness, the size of the entire heart rate module may be affected.

The heart rate module of the present disclosure includes two optical sensor chips, namely the first optical sensor chip 7 and the second optical sensor chip 8. In the case of an effective module size, by adjusting the distance between the first light wave emitting unit and the first optical sensor chip 7, as well as the distance between the second light wave emitting unit and the second optical sensor chip 8, the first optical sensor chip 7 can better receive the red light waves and infrared light waves reflected from the skin or blood, and the second optical sensor chip 8 can better receive the green light waves reflected from the skin or blood. That is, by adjusting the positions of the two optical sensor chips, a better optical distance can be achieved, which is helpful to realize accurate detection.

Wherein, the distance between the first optical sensor chip 7 and the first light wave emitting unit should be controlled within 6 to 10 mm, and when the distance is close to 9 mm, the receiving effect for the red light waves and infrared light waves is the best. The distance between the second optical sensor chip 8 and the second light wave emitting unit should be controlled at about 2.3 mm to 3.2 mm, and when the distance is 2.5 mm, the receiving effect for the green light waves is the best.

In a specific embodiment of the present disclosure, referring to FIG. 1, on a substrate, the distance between the first optical sensor chip 7 and the first light wave emitting unit is controlled to be 9 mm. At this time, there is a certain free space between the first optical sensor chip 7 and the first light wave emitting unit. In consideration of reducing the size of the entire heart rate module, this space can be effectively used to install the second light wave emitting unit and the second optical sensor chip 7. In order to prevent the light waves emitted by the first light wave emitting unit from interfering with the light waves emitted by the second light wave emitting unit, the second light wave emitting unit may be provided close to the first optical sensor chip 7, and the second optical sensor chip 8 may be close to the first light wave emitting unit, so that the first light wave emitting unit and the second light wave emitting unit are separated. Wherein it is necessary to ensure that the distance between the second optical sensor chip 8 and the second light wave emitting unit is 2.5 mm. By adopting this design, the distance between the green light waves and the red light waves/infrared light waves and the corresponding optical sensor chip can be adjusted under the condition of a limited module size. In this way, the present disclosure achieves a better optical distance and finally achieves good detection results.

In another specific embodiment of the present disclosure, on a substrate, the distance between the first optical sensor chip 7 and the first light wave emitting unit is 9 mm (this distance is the distance that can optimally receive the reflected red light waves and infrared light waves); the second light wave emitting unit and the second optical sensor chip 8 are provided between the first optical sensor chip 7 and the first light wave emitting unit, wherein the second light wave emitting unit is provided close to the first optical sensor chip 7, and the second optical sensor chip 8 is provided close to the first light wave emitting unit; and the distance between the second optical sensor chip 8 and the second light wave emitting unit is 2.5 mm, and the distance between the first optical sensor chip 7 and the second light wave emitting unit is also 2.5 mm. This design enables the first optical sensor chip 7 to receive both the red light waves and infrared light waves reflected from the skin or blood, and the green light waves reflected from the skin or blood, and the second optical sensor chip 8 can receive the green light waves reflected from the skin or blood. The method of averaging after multiple calculations is beneficial to improve the accuracy of the detection results.

It should be noted that the positions of the first light wave emitting unit, the second light wave emitting unit, the first optical sensor chip 7, and the second optical sensor chip 8 on the substrate are not limited to the above two embodiments, but can be adjusted flexibly based on the size of the heart rate model, as long as the light waves can be received, which is not limited in the present disclosure.

The heart rate module of the present disclosure, as shown in FIG. 1, further includes the analog front end 3 (AFE) and the power management unit 4 (BOOST) provided on the substrate. Wherein, the analog front end 3 and the power management unit 4 are both mounted on the substrate and connected to the circuit in the substrate. The first optical sensor chip 7 and the second optical sensor chip 8 are mounted on the power management unit 4 and the analog front end 3 respectively.

In general, the heart rate module of the present disclosure is measured in a manner close to the human skin. Wherein, the power management unit 4 can control the first light wave emitting unit (the two red LED chips 1 and one infrared LED chip 2) and the second light wave emitting unit (the three green LED chips 5) to emit light, and the light emitted by the first light wave emitting unit and the second light wave emitting unit is directed to the human skin, and a part of the light will be absorbed by skin soft tissues while the other part is reflected back from the skin or blood and received by the first optical sensor chip 7 and the second optical sensor chip 8 respectively. For example, a difference in oxygen content in the blood will cause a difference in absorption rate of the red light and the infrared light, which will cause the reflected light to have a slight change and cause output current of the first optical sensor chip 7 to change. This change is converted by the analog front end (AFE) 6 and sent to other components such as a processor for further processing, for example, by comparing the intensity of the red light signal with that of the infrared light signal, so as to calculate the oxygen content in the blood, that is, obtain a blood oxygen value. Moreover, when the heart beats, blood will flow in the skin, which will cause changes in the blood content in the skin, and then by calculating the relationship between changes of the red light signal or infrared light signal and time, the heart rate can be calculated. In addition, the heart rate data can also be tested by matching the green LED chip 5 with the second optical sensor chip 8 to measure the change of blood content, and then calculating the relationship between the change of the green light signal and time.

The heart rate module of the present disclosure reduces unnecessary complex structural design, and can package various components together in a simple manner, reduce the space occupied by each component of the whole machine, and reduce the volume of the heart rate module, that is, realizing the miniaturization of the heart rate module. Moreover, the power supply is reasonably distributed through the power management unit 4, which increases the standby time of the battery in the heart rate module. At the same time, the heart rate module of the present disclosure also solves the problem of gathering the light of the LED of the heart rate module, effectively overcomes the defects in the prior art, and helps to improve the accuracy of the heart rate detection results.

On the other hand, an embodiment of the present disclosure also provides an electronic device for collecting heart rate, and the electronic device includes any of the above-mentioned heart rate modules.

Wherein, the electronic device for collecting the heart rate of the present disclosure may be an electronic product such as smart bracelets, smart watches, smart phones, and portable medical devices, which is not limited in the disclosure. The electronic device for collecting the heart rate of the present disclosure can be used for detecting the heart rate and have the advantage of accurate detection results.

While certain specific embodiments of the present disclosure have been illustrated by way of example, it will be understood by those skilled in the art that the foregoing examples are provided for the purpose of illustration and are not intended to limit the scope of the present disclosure. It will be understood by those skilled in the art that the foregoing embodiments may be modified without departing from the scope and spirit of the disclosure. The scope of the present disclosure is subject to the attached claims.

1. A heart rate module, comprising a substrate, a first light wave emitting unit, a second light wave emitting unit, a first optical sensor chip, and a second optical sensor chip provided on the substrate;
the substrate is provided thereon with an isolation grating wall separating the first light wave emitting unit, the second light wave emitting unit, the first optical sensor chip, and the second optical sensor chip from each other;
the isolation grating wall together with the substrate form a first accommodating cavity for accommodating the first light wave emitting unit, a second accommodating cavity for accommodating the second light wave emitting unit, a third accommodating cavity for accommodating the first optical sensor chip, and a fourth accommodating cavity for accommodating the second optical sensor chip;
wherein each of the first accommodating cavity and the second accommodating cavity have a pattern draft on peripheral inner walls thereof, and are provided on at least a part of the inner walls thereof with non-conductive reflective coatings.

2. The heart rate module according to claim 1, wherein the reflective coatings are made of Ti₃0₅ material or SiO₂ material or non-conductive high-aluminum reflective film.

3. The heart rate module according to claim 1, wherein the reflective coatings have a thickness no greater than 1 μm.

4. The heart rate module according to claim 1, wherein the pattern draft is ≥45°.

5. The heart rate module according to claim 1, wherein the first optical sensor chip and the first light wave emitting unit have a distance of 6 to 10 mm therebetween; and
the second optical sensor chip and the second light wave emitting unit have a distance of 2.3 to 3.2 mm therebetween.

6. The heart rate module according to claim 1, wherein the first optical sensor chip and the first light wave emitting unit have a distance of 9 mm therebetween;
the second light wave emitting unit and the second optical sensor chip are provided between the first optical sensor chip and the first light wave emitting unit, the second light wave emitting unit being close to the first optical sensor chip, the second optical sensor chip being close to the first light wave emitting unit, and the second optical sensor chip and the second light wave emitting unit having a distance of 2.5 mm therebetween.

7. The heart rate module according to claim 1, wherein the first light wave emitting unit comprises two red LED chips and one infrared LED chip, wherein the two red LED chips and the one infrared LED chip are distributed in a straight line, and the infrared LED chip is located between the two red LED chips.

8. The heart rate module according to claim 1, wherein the second light wave emitting unit comprises three green LED chips which are distributed in a straight line.

9. The heart rate module according to claim 1, wherein the isolation grating wall is further provided thereon with light-transmitting windows that allow the first accommodating cavity, the second accommodating cavity, the third accommodating cavity, and the fourth accommodating cavity to be in communication with the outside.

10. The heart rate module according to claim 1, further comprising an analog front end and a power management unit provided on the substrate;
the first optical sensor chip and the second optical sensor chip are respectively mounted on the power management unit and the analog front end.

11. An electronic device for collecting heart rate, comprising the heart rate module according to claim 1.
